(19) United States
(12) Patent Application Publication
(10) Pub. No.: US 2009/0110723 A1
(43) Pub. Date: Apr. 30, 2009

(54) Linkers for multipart dosage forms for release of one or more pharmaceutical compositions, and the resulting dosage forms

(76) Inventor: Stephen Mark McAllister, Sandwich (GB)

Correspondence Address: Glaxo Smith Kline/Finnegan, Henderson LLP, 901 New York Avenue, NW, Washington, DC, 20001, US

(21) Appl. No.: 12/285,844

(22) Filed: Oct. 15, 2008

Related U.S. Application Data
(60) Provisional application No. 60/960,786, filed on Oct. 15, 2007.

(51) Int. Cl. A61K 9/48

(52) U.S. Cl. 424/454

(57) ABSTRACT

A linker for multicomponent dosage form has a preformed drug substance tablet and an injection molded jacket including one or both axial jacket ends open for dispensing the preformed drug substances. The jacket has snap-fit connection elements on the outer surface adjacent each end for engagement with complementary snap-fit element on capsule-type and cap-type dosage form units. Another multicomponent drug form linker is injection molded with a raised circumferential band and snap-fit elements between the band and respective ends engageable with complementary snap-fit elements on dosage form units to provide compressive abutting contact between ends of the units and the band. The band can have an axially tapered outer surface to accommodate dosage form units of different diameters. Still another multicomponent dosage form linker has separable male and female parts each engageable with, and separately sealing, respective dosage form units to be linked. Circumferential complementary grooves, groove segments, ridges and ridge segment-type snap-fit elements are disclosed, as are dosage forms using the above-described linkers.

### PRIORITY

This application claims priority to U.S. Provisional Patent Application No. 60/960,786 filed Oct. 15, 2007.

### FIELD OF THE INVENTION

This invention relates to pharmaceutical dosage forms and, more particularly, to multipart capsules including a linker unit and one or more connected sub-units for oral dosing.

### BACKGROUND OF THE INVENTION

Various types of pharmaceutical dosage forms are known for oral dosing. Such capsules generally comprise an envelope wall of a pharmaceutically acceptable, e.g. orally ingestible, polymer material such as gelatin, although other materials for capsule walls, e.g. starch and cellulose based polymers are also known. Such capsules generally have soft walls made by forming a film on a capsule former, which is then allowed to dry. Rigid walled capsules made by injection molding are also known; see for example U.S. Pat. No. 4,576,284, U.S. Pat. No. 4,591,475, U.S. Pat. No. 4,655,840, U.S. Pat. No. 4,738,724, U.S. Pat. No. 4,738,817, and U.S. Pat. No. 4,790,881 (all to Warner Lambert). These disclose specific constructions of capsules made of gelatin, starch and other polymers, and methods of making them by injection molding of hydrophilic polymer, e.g., water mixtures. U.S. Pat. No. 4,576,284 specifically discloses such capsules provided with a cap which closes the capsule and is formed in situ on the filled capsule by molding. U.S. Pat. No. 4,738,724 discloses a wide range of rigid capsule shapes and parts.

Multi-compartment capsules, including those of the type where each compartment has different drug release characteristics or, for example, contains a different drug substance or formulation, are also known; see for example U.S. Pat. No. 4,738,724 (Warner-Lambert), U.S. Pat. No. 5,672,359 (University of Kentucky), U.S. Pat. No. 5,443,461 (Alza Corp.), WO 9516438 (Cortecs Ltd.), WO 9012567 (Helminthology Inst.), DE-A-3727894, BE 900950 (Warner Lambert), FR 2524311, NL 7610038 (Tapanhony Nev.), FR 28646 (Pluripharm), and U.S. Pat. No. 3,228,789 (Glassman), U.S. Pat. No. 3,186,910 (Glassman), among others. U.S. Pat. No. 4,738,817, U.S. Pat. No. 3,228,789, and U.S. Pat. No. 3,186,910 each disclose a multicompartment capsule made of a water-plasticized gelatin.

Pharmaceutical dosage forms that comprise a matrix of a solid polymer, in which a drug substance is dispersed, embedded or dissolved as a solid solution are also known. Such matrixes may be formed by an injection molding process. This technology is discussed in Cuff G. and Raouf F., Pharmaceutical Technology, June 1998, p. 96-106. Some specific formulations for such dosage forms are, for example disclosed in U.S. Pat. No. 4,678,516; U.S. Pat. No. 4,806,337; U.S. Pat. No. 4,764,378; U.S. Pat. No. 5,004,601; U.S. Pat. No. 5,135,752; U.S. Pat. No. 5,244,668; U.S. Pat. No. 5,139,790; U.S. Pat. No. 5,082,655 among others, in which a polyethylene glycol (“PEG”) matrix is used and solid dosage forms are made by injection molding.

The content of the above-mentioned background patent publications is incorporated herein by way of reference.

See, also for example, WO 01/08666, WO 02/060385, US 2004/0115256, US 2006/0049311, WO 02/060384, US 2003/0068369, US 2004/0166153, WO 04/010978, US 2006/0057201, WO 05/009380, US 2005/0175687, WO 05/089726, US 2005/0249807, U.S. 60/968,383, and U.S. 61/061,275, each of the disclosures of which are incorporated herein by way of reference.

Also, the content of PCT/EP00/07295 entitled “MULTI-COMPONENT PHARMACEUTICAL DOSAGE FORM” assigned to the assignee of the present application, is incorporated herein by way of reference.

### SUMMARY OF THE INVENTION

In one aspect of the present invention, a linker for connecting to one or more dosage form units from the group including capsule compartments and closure caps includes a solid drug substance in tablet form, the tablet having a longitudinal axis and being substantially cylindrical with opposed axial end faces. The linker also includes a jacket formed around and radially confining the tablet. The jacket has an outer wall with longitudinal ends, one or both of the jacket ends being open for dispensing the drug substance from the respective end faces. The jacket outer wall further has snap-fit elements located adjacent one or both longitudinal ends. Preferably, the snap-fit elements are selected from circumferential grooves, groove segments, ridges, and ridge segments.

In another aspect of the present invention, a multicomponent dosage form includes a linker unit having a solid drug substance in tablet form disposed within a jacket, the tablet having a longitudinal axis and being substantially cylindrical with opposed longitudinal end faces. The jacket radially confines the tablet and has an outer wall with longitudinal ends and a raised circumferential band between the jacket ends. The jacket outer wall also has a snap-fit element located adjacent one longitudinal end. The multicomponent dosage form also includes at least one dosage form unit selected from the group including capsule compartments and closure caps, the dosage form unit having an open end connected to the one jacket longitudinal end of the linker unit. The dosage form unit has a snap-fit element complementary to and configured to engage the snap-fit element of the one jacket longitudinal end.

In a further aspect of the present invention, a linker for connecting two dosage form units from the group including capsule compartments and closure caps each having an open end, includes a substantially cylindrical tube having an interior and a longitudinal axis, the tube also having an outer surface and opposed axial ends. The linker also has a raised band positioned on the tube outer surface, the band being located between the opposed axial tube ends and extending circumferentially around the tube outer surface. The raised band has opposed side surfaces configured for compressive abutting contact with the open ends of the dosage form units. Snap-fit elements are located on the tube outer surface between the raised band and a respective one of the tube axial ends. Preferably, the snap-fit elements are selected from circumferential grooves, groove segments, ridges and ridge segments. The linker still further includes a wall configured to close the tube interior, the wall being positioned along the axis at a location between and including the axial tube ends.

In a still further aspect of the present invention, a dosage form includes a linker having a substantially cylindrical tube with an interior and a longitudinal axis, the tube also has an outer surface and opposed axial ends. The linker also has a raised band positioned on the outer surface, the raised band being located between the opposed axial tube ends and extending circumferentially around the tube outer surface and having opposed side surfaces oriented substantially perpendicular to the axis. The linker also has snap-fit elements located on the tube outer surface between the raised band and each of the tube axial ends and a wall configured and dimensioned to close the tube, the wall being positioned along the axis at a location between and including the axial tube ends. The dosage form further includes a pair of dosage form units selected from capsule compartments and closure caps connected to the linker axial ends, each of unit having a complementary snap-fit element formed on a respective inner unit surface adjacent a dosage form unit open end and being engaged with a respective snap-fit element on the linker. The configuration of the snap-fit elements and spacing from the raised band on the linker and the configuration of the complementary snap-fit elements and spacing from the open ends on the respective dosage form units provides a compressive abutting contact between the open ends of the units and the respective sides of the raised band.

In yet another aspect of the present invention, a linker for connecting and separately sealing first and second capsule compartments, includes a first generally cylindrical linker part having an axis and opposed longitudinal ends. The first part also has an outer surface with a diameter, an inner surface with a diameter, and a closed wall, the outer surface including a first snap-fit element configured for engaging a complementary snap-fit element on the first capsule compartment, and the inner surface including another snap-fit element. The linker also includes a second generally cylindrical linker part having an axis, opposed longitudinal ends, and a closed wall. The second part also has a large diameter portion adjacent one second part end and a small diameter portion adjacent and extending axially from the other second part end, the large and small diameter portions having respective outer surfaces. The outer surface of the large diameter portion has a second snap-fit element configured for engaging a complementary snap-fit element on a second capsule compartment. Preferably, the first and second snap-fit elements and the another snap-fit element are selected from circumferential grooves, groove segments, ridges, and ridge segments. Also, the outer surface of the small diameter portion of the second part contains a further snap-fit element, preferably selected from circumferential grooves, groove segments, ridges, and ridge segments complementary to the another snap-fit element on the inner surface of the first linker part. The another snap-fit element and further snap-fit element are engageable to releasably connect the first and second linker parts, and the first and second closed end walls are configured to seal off respective ones of the two capsule compartments when the linker parts are engaged with the respective capsule compartments.

And in still yet another aspect of the present invention, a dosage form includes a first generally cylindrical capsule compartment having an axis, a closed end, and an open end, the open end including an integral, annular linker member extending axially outward of the open end and having an outer surface with a first snap-fit element located a distance L₁ from the capsule compartment open end. The dosage form also includes a second generally cylindrical capsule compartment having an axis, a closed end, and an open end with an inner surface, the second capsule compartment having a second snap-fit element complementary to the first snap-fit element, the second snap-fit element being located on the inner surface and spaced a distance of L₂ from the second capsule open end. L₂ is greater than L₁, and the first and second snap-fit elements are engageable to connect the first and second capsule compartments with the respective open ends in compressive abutting contact with each other.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

The invention will now be described by way of example with reference to:

FIG. 1 which is a perspective view of a linker made in accordance with one aspect of the present invention;

FIG. 2 is a cross section of a preformed tablet component of the linker depicted in FIG. 1;

FIG. 3 shows various perspective views of the preformed tablet component shown in FIG. 2;

FIG. 4 is a cross sectional view of the linker shown in FIG. 1 and having a closing wall at one end;

FIG. 5 shows various perspective views of the linker shown in FIG. 4;

FIG. 6 is an exploded view of a multipart dosage form utilizing the linker shown in FIGS. 1 and 2;

FIG. 7 is a cross sectional view of an assembled dosage form including the linker shown in FIG. 1;

FIG. 8 is an enlarged view of detail A in FIG. 7;

FIG. 9 shows an alternative construction of the groove-type snap-fit element shown in FIG. 8;

FIG. 10 is an exploded view of another embodiment of the present invention;

FIG. 11 shows various perspective and end views of the dosage form of FIG. 10 in an assembled state;

FIG. 12 depicts an exploded cross sectional view of the dosage form shown in FIG. 10;

FIG. 13 is a cross sectional view depicting a linker form in accordance with yet another aspect of the present invention;

FIG. 14 shows assembled and disassembled dosage forms using the linker shown in FIG. 13;

FIG. 15 is a cross sectional view of a variation of the linker unit shown in FIG. 13 connected to capsule compartment-type dosage form units;

FIG. 16 is an exploded cross sectional view of a dosage form utilizing a further variation of the linker shown in FIG. 13;

FIG. 17 illustrates dosage form, dosage form parts, and linker parts made in accordance with a yet further aspect of the present invention;

FIG. 18 is an exploded cross sectional view of the two-part linker as shown in FIG. 17;

FIG. 18A is an end view of the female portion of the two-part linker shown in FIG. 18 taken in the direction AA; and

FIG. 19 is an exploded cross sectional view of a dosage form utilizing the linker shown in FIGS. 18 and 18A.

### DETAILED DESCRIPTION

Reference will now be made in detail to the present embodiments of the invention, examples of which are illustrated in the accompanying drawings.

In accordance with one aspect of the present invention, a linker is disclosed for connecting with one or more dosage form units from the group including capsule compartments and closure caps, with the linker holding a drug substance. Specifically, the linker includes a solid drug substance in tablet form, the tablet having a longitudinal axis and being substantially cylindrical with opposed axial end faces. The linker further includes a jacket formed around and radially confining the tablet, the jacket having an outer wall with longitudinal ends, one or both of the jacket ends being opened for dispensing the drug substance from the respective end face. The linker still further includes the jacket outer wall a having snap-fit element adjacent at least one jacket longitudinal end, the snap-fit element preferably being selected from circumferential grooves, groove segments, ridges, and ridge segments.

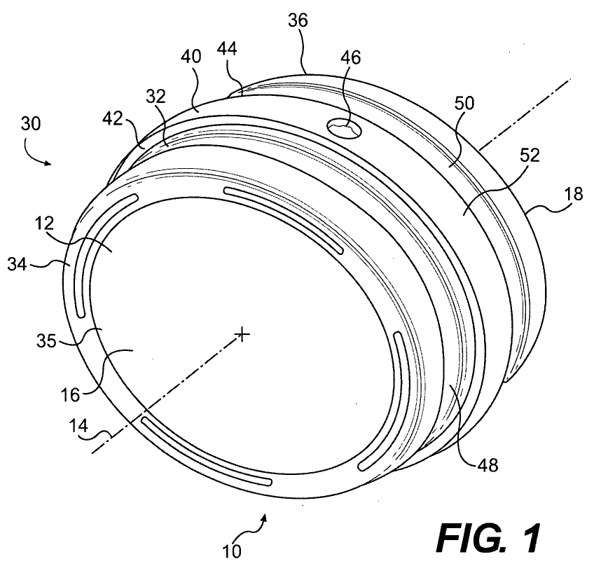

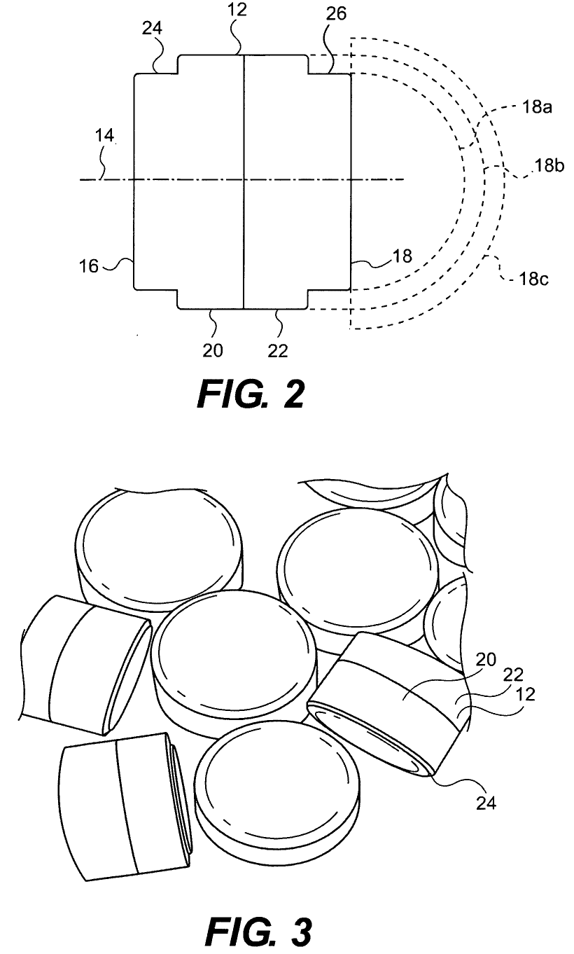

As embodied herein and with initial reference to FIGS. 1-4, linker 10 includes tablet 12, which may be substantially cylindrical, having an axis 14 and closed axial end faces 16 and 18. End faces 16 and 18 may be generally planar and perpendicular to axis 14, or one or both may be shaped to extend axially, for reasons to be discussed below, such as rounded end face 18a, 18b, or 18c depicted by dotted lines in FIG. 2. Tablet 12 preferably is preformed outside linker 10 by processes such as dry compacting, casting, or other process known in the art. Tablet 12 can be composed of a single drug substance or, as shown in FIG. 2, a bi-layer configuration can be used having drug substance parts 20, 22. The drug substances in parts 20 and 22 may differ in composition and/or release characteristics, and can be appropriately indicated as such by coloration to ensure correct assembly into dosage forms, as will be discussed below. Also, it is preferred that tablet 12 includes recessed lands 24, 26 formed around respective perimeters of tablet end faces 16, 18, for securing jacket 30 of linker 10 as will be discussed below.

Drug substances for use in dosage forms suitable for being administered orally to a patient can be included in a linker 10 and, in particular, that can be used in parts 20 and 22, can include any suitable or conventional form, such as, for example, a powder, granules, compact, microcapsules, gel, syrup, or liquid, provided that the capsule portion wall material is sufficiently inert to the liquid content of the latter three forms.

As embodied herein and with continued reference to FIGS. 1, 4 and 5, linker 10 includes a jacket 30 with a generally cylindrical outer wall 32 and respective longitudinal ends 34, 36. Jacket 30 may be constructed by injection molding around tablet 12 in order to leave one or both jacket ends 34, 36 open and to expose tablet end face 16 and/or 18 for dispersion and dissolution of the contained drug substance(s) once a connected capsule and/or cap dosage form unit has been breached, such as, for example, by changing shape, form, or structure within a gastrointestinal environment, e.g., dispersing, dissolving, disintegrating, swelling, being partially or completely soluble, or otherwise changeable when exposed to stomach pH and/or in intestine pH. It is contemplated that jacket 30 may be injection molded around tablet 12 and that tablet 12 may be supported within a mold by, for example, one or more finger or gripper elements extending outward from a mold wall into the interior of the mold and engaging a portion of tablet 12, e.g., engaging the radially outer wall of tablet 12.

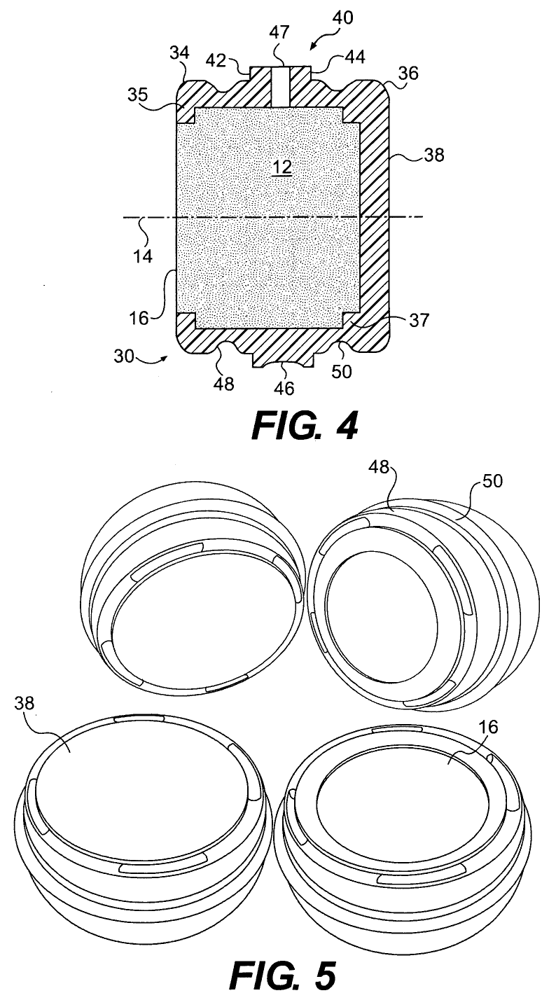

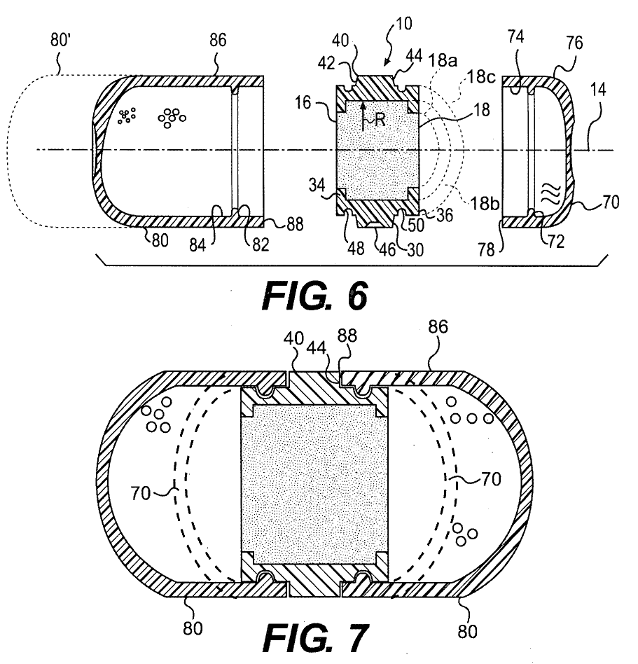

For example, in linker 10, shown in cross-section in FIG. 6, both jacket ends 34, 36 are open to expose tablet end faces 16, 18 for drug substance dissolution and/or dispersion, when cap unit 70 and/or capsule unit 80 are breached. In comparison, FIGS. 4 and 5 show a variation in jacket 30 with a solid wall 38 closing off jacket end 36, to substantially prevent the drug substance in tablet 12 from dissolving/dispersing therethrough. Wall 38 can be integrally formed with the remainder of jacket 30 via injection molding. In both variations the injection molded jacket material radially confines a tablet 12 (radial direction being depicted in FIG. 6 as indicated by arrow R) and also axially confines tablet 12 to at least some degree as a result of jacket material flowing into the recess lands 24, 26 of tablet 12 and forming circumferential jacket end edges 35, 37. It is contemplated that a radially confining injection molded jacket includes the jacket surrounding at least a portion and contacting at least a portion of the circumference of the tablet, and may, for example, include the jacket being injection molded around the tablet or other method providing a jacket radially confining the tablet. It is also contemplated that slots 90 may be formed in jacket 30 at one or both of jacket ends 34, 36 due to tablet 12 being supported within a mold by the fingers or grippers (discussed above) during injection molding and which may aid in dissolution/dispersion of the tablet. See FIGS. 1 and 5. Although four slots are illustrated in jacket end 34 (see FIG. 1), it is contemplated that any number of slots may be formed.

As further embodied herein, and referring again to FIG. 1, jacket 30 includes a raised band 40 circumferentially formed on the periphery of outer jacket wall 32, preferably midway between longitudinal ends 34 and 36. Band 40 includes opposed side surfaces 42, 44 configured for abutting contact with the wall ends of dosage form units, e.g., cap unit 70 and/or capsule unit 80, interconnected with linker 10. As depicted in FIGS. 1 and 4-6, side surfaces 42, 44 may be essentially perpendicular to axis 14 and raised band 40 also can have one or more concave depressions 46 to accommodate an injection molding feedgate. Also, as shown in FIG. 4, raised band 40 may have one or more radially directed apertures 47 to provide a direct path for controlled, relatively early dispersion of the drug substance in tablet 12, prior to breach or dissolution of any cap or capsule covering exposed tablet end faces, such as end face 16 in FIG. 4. Apertures 47 may be sealed with a rapidly dissolving thin film or coating (not shown) to prevent contamination of the drug substance of tablet 12. Such a film or coating may be made from any Phama-acceptable polymer suitable for film coating.

As further embodied herein, jacket 30 includes snap-fit elements 48, 50 formed on outer surface 52 of jacket wall 32 between raised band 40 and respective jacket longitudinals ends 34, 36. As shown in FIGS. 1 and 4, both snap-fit elements 48, 50 are circumferential grooves configured and dimensioned to engage complementary circumferential ridge or “bead” elements on the inner surfaces of the respective cap unit or capsule unit. For example, cap unit 70 shown in FIG. 6 has a continuous circumferential ridge or bead 72 formed on inner surface 74 of outer wall 76 adjacent wall end 78. Ridge 72 is configured to engage groove 50 in linker 10 by a “snap-fit inter-connection.” By snap-fit inter-connection, it is meant that the resiliency of cap unit 70 expands to allow ridge 72 to pass over a jacket surface 52 and subsequently contracts to allow ridge 72 to engage or “snap” into groove 50.

Similarly, and as shown in FIG. 6, capsule unit 80 has a circumferential ridge 82 on inner surface 84 of capsule unit wall 86 adjacent wall end 88 configured and dimensioned to be complementary to groove 48 of linker 10 provide a snap-fit inter connection.

Referring to FIGS. 6 and 7, it can be seen that linker 10 may make possible multiple dosage form configurations interconnected with a snap-fit connections, including two capsule units 80 joined by linker 10 (see FIG. 7); two cap units 70 joined by linker 10 (see FIG. 7 shown as dashed); or a cap unit 70 joined by linker 10 to a capsule unit 80 (see FIG. 6). However, linker 10 can be used with only a single joined capsule, such as capsule 80 in the embodiment depicted in FIG. 6 but without cap 70, such as if the linker 30 is configured to have a solid end wall, such as end wall 38 in the FIG. 4 embodiment. Moreover, if dissolution/dispersion of an increased quantity of the solid drug in linker 30 is desired, the dosage form in FIG. 6 may be used without end cap 70 with the end face 18 drug material extended axially past linker end. In such a variation, the tablet end face 18 also may be shaped to provide easier swallowing such as with a rounded, extended face such as 18a depicted with dashed line in FIG. 6. Other configurations of an extended tablet end face that may be desirable include those depicted in FIG. 6 as 18b and 18c. Extended end face 18b may be achieved by omitting recess 26 in tablet 12 (see FIG. 2) and jacket end edge 37 (see FIG. 4) and tapering the jacket and edge 36 to provide a smoother dosage form end. A “mushroom” shaped extended end face configuration in 18c is an alternative way to achieve a smooth dosage form end. Also, “exposed” tablet end face 18a, 18b, or 18c may be covered with a thin rapidly dissolving film or coating (not shown) to prevent contamination of the drug substance of tablet 12. Such a film or coating may be made from any Phama-acceptable polymer suitable for film coating.

Further, in each of the above-discussed variants where linker end 36 is to be left uncapped, a snap-fit connection adjacent linker end 36 such as groove 50 may not be needed and band 40 tapered axially, thereby possibly simplifying the construction and manufacture of the linker. However, if linker 30 is constructed without a jacket end edge, such as for extended tablet end faces 18b and 18c, then it may be desirable to use a film covering (not shown) at least over the extended tablet face and adjacent linker end 36 to help axially constrain tablet 18.

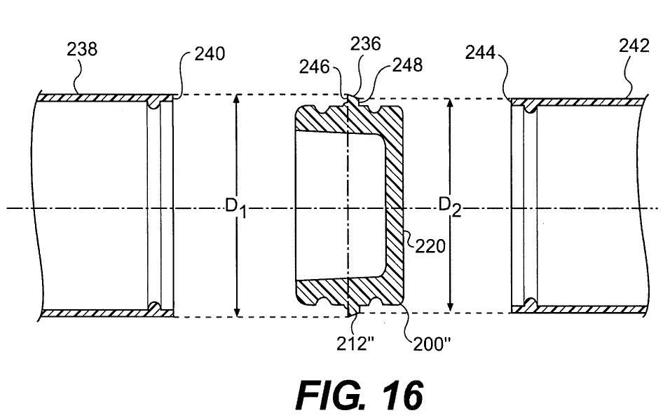

FIG. 6 also depicts the use of capsules of varying length, e.g., a longer length capsule 80′ shown as dashed, making possible asymmetric dosage forms. Moreover, while FIGS. 1 and 4-7 show substantially symmetrical linkers having outer wall diameters at longitudinal ends 34 and 36 substantially equal and configured for attaching capsule and/or cap units of essentially the same diameter, variations in linker 10 having different jacket wall diameters at ends 34 and 36 are contemplated to interconnect capsule units and/or cap units of different diameters, thereby making possible a dosage form asymmetric in width. See also discussion of linker 212″ shown in FIG. 16 described below.

Moreover, it is contemplated that the dimensions of the capsules, end caps, and linkers along with the respective snap-fit elements, e.g., cap unit 70 and linker 30, including respective ridge 72 and groove 50 in FIG. 6, may be adjusted to achieve any degree of snap-fit and that the snap-fit is preferably a relatively permanent, locking interconnection therebetween. That is, although the parts could be separated with sufficient force, such separation is not intended in the normal course of utilization of the dosage forms by a consumer. It is also contemplated that the position of the groove and ridge elements could be reversed, and that complementary ridge segments and groove segments could be used instead of circumferentially continuous members. Also, although ridge segments could be used with continuous grooves, leakage of the contained drug substances might possibly occur. Further, although the complementary grooves and ridges/“beads” shown in FIGS. 1 and 4-7 are generally rounded or circular in axial cross section, other shapes such as, for example, wedges, trapezoids, triangles, may be used.

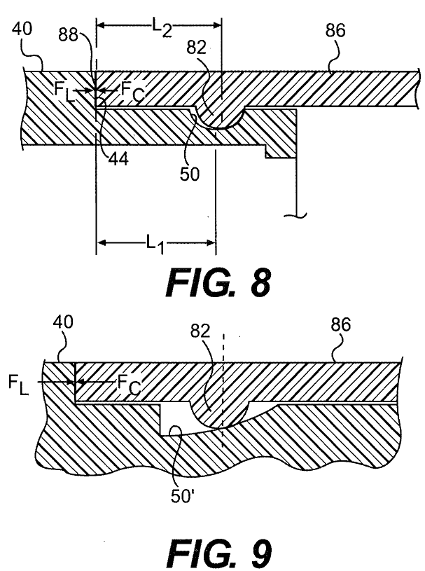

It is preferred that the linker, capsule units, and cap units be configured and dimensioned along with the snap-fit elements to provide a compressively abutting contact between the linker band 40, particularly band side surfaces 42, 44, and wall ends 78 and 88 of capsule unit 70 and cap unit 80, respectively. As best seen in FIG. 8, which is an enlarged view of detail A of FIG. 7, the distance L₁ between ridge 82 and the capsule wall end 88 may be slightly larger than the distance L₂ between groove 50 and band side 44 of linker 10. As such, upon engagement between the capsule wall end 88 and band side 44, the ridge 82 is not “fully seated” in groove 50. But as a consequence of the resiliency of cap end 86 an axial force tending to fully seat ridge 82 results in a force F_{c} exerted by a capsule wall and 88 against a linker band side 44 and a similar force F_{L} opposing this force. This compressively abutting cap contact may provide increased dosage form integrity, because it may eliminate any gap between the raised band 40 and the capsule end wall 88 which might otherwise establish a groove, edge, or other surface inconsistency that might increase a tendency to disengage the capsule unit from the linker. Also, the compressive abutting contact may minimize the chance for radial movement between the capsule unit and the linker and provide a relatively smooth surface to be established across the contact therebetween, potentially facilitating easier swallowing.

Still further, the compressive abutting contact provided by band 40 might provide a further barrier as a mechanical seal against unwanted drug substance leakage from the capsule compartments or the linker. This would be in addition to the seal provided by the contact between the snap-fit elements on the linker and the capsule compartments and/or closure caps. Also, the components of the multicompartment dosage forms can be configured to have a slight interference fit between the outer surface of the linker component and the inner surfaces of the capsule and/or cap units, to provide yet another barrier against leakage, while still providing ease of assembly.

Other means for providing a compressive abutting contact between the capsule and cap units against the linker band are contemplated including the use of different cross-sectional geometries of the grooves and/or ridges, as stated previously. Such a variation is shown in FIG. 9 where a wedge-shaped groove 50′ is used instead of a circular cross-section. Again, it is contemplated that the one skilled in the art given this configuration and dimensions the linkers, capsules and/or cap units and associated snap-fit elements may be adjusted to achieve the desired compressive abutting contact function.

In accordance with another aspect of the present invention, a dosage form incorporating a linker component includes a first generally cylindrical capsule compartment having an axis, a closed end, and an open end. The open end includes an integral, annular linker member extending axially outward of the open end. The linker member has an outer surface with a first snap-fit element located a distance L₁ from the capsule compartment open end. The dosage form further includes a second generally cylindrical capsule compartment having an axis, a closed end, and an open end with an inner surface. A second snap-fit element complementary to the first snap-fit element is provided on the inner surface and spaced a distance of L₂ from the second capsule open end. L₂ is configured to be greater than L₁, and the first and second snap-fit elements are engageable to connect the first and second capsule compartments with respective open ends being in compressive abutting contact with each other.

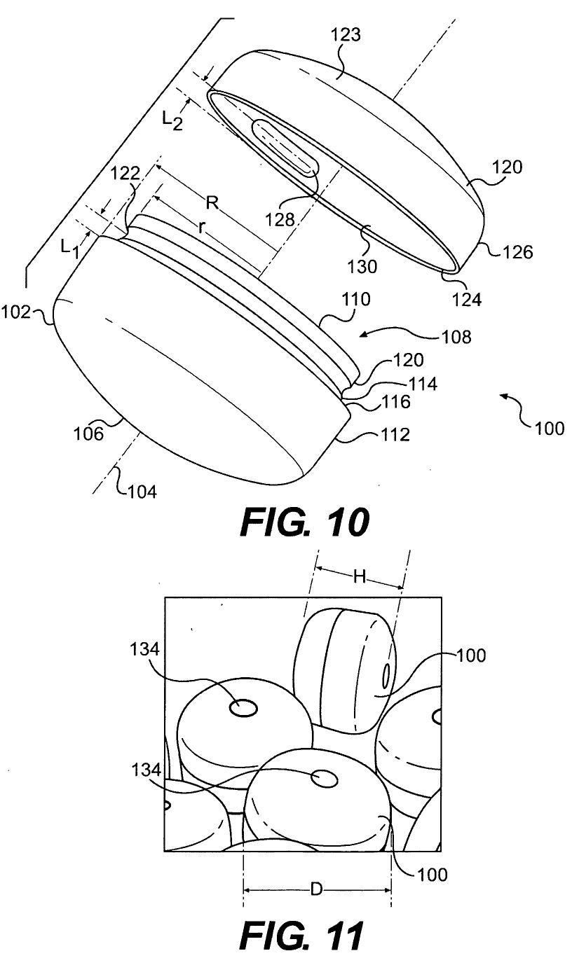

As embodied herein, FIGS. 10-12 illustrate a dosage form, designated generally as 100, that includes a first generally cylindrical capsule compartment 102 having axis 104. First capsule compartment 102 includes closed end 106 and open end 108. First capsule compartment 102 further includes annular linker member 110 fixed to an inner part 114 of the capsule compartment wall 112 and extending in the axial direction beyond outer wall 116, in a direction away from closed end 106. Linker member 110 is integral with first capsule compartment 102, such as, for example, by being consanguineous, by being injection molded together, being fused or welded, and/or connected via any other suitable method for integrally forming linker member 110 and first capsule compartment 102. Integral, as used herein, is intended to include substantially permanent connections, e.g., welds, between two elements and is not intended to include releasable connections, e.g., snap-fit connections, between two elements.

Linker member 110 further includes an outer surface 120 having a radius “r” less than the radius “R” of outer wall 112, as illustrated in FIG. 10. Circumferential groove 122 is formed on surface 120 at a distance L₁ from the end of outer wall 116, to act as a snap-fit element.

Dosage form 100 further includes a second capsule compartment 120 which is also generally cylindrical about axis 104 with closed end 123, open end 124, and outer wall 126. Three ridge segments 128 (only one being shown in FIG. 10) are formed on inner surface 130 of wall 126 circumferentially spaced about axis 104, to function as a complementary snap-fit element for engaging groove 122 of first capsule compartment 102.

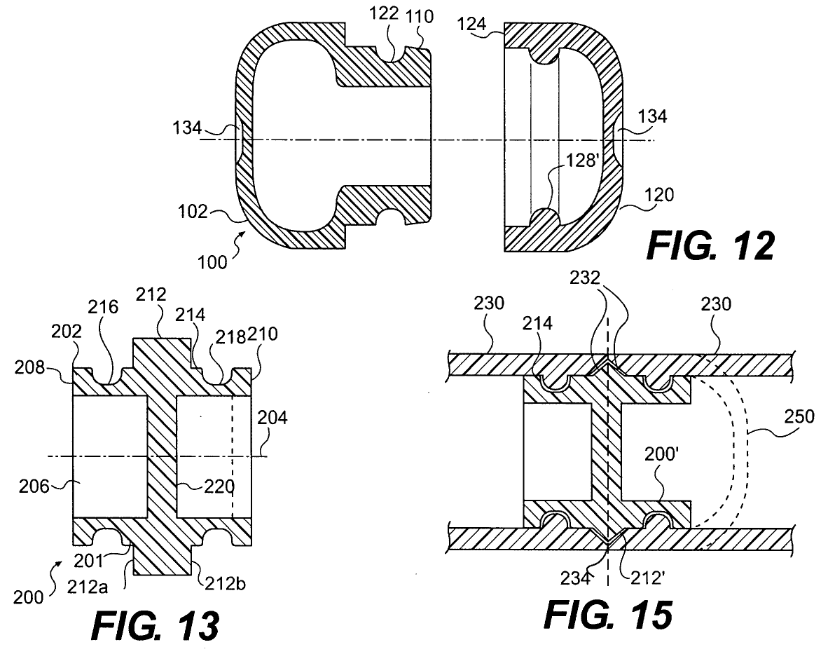

The dosage form 100 shown in FIG. 11 is configured as a tablet and has a height/diameter ratio less than or equal to 1.0. In order to ensure a continuous overall dosage form outer surface, the capsule compartments and linker member, including the respective snap-fit elements (groove 122 and ridge segments 128) are configured and dimensioned to achieve compressive abutting contact between outer wall part 116 of compartment 102 and outer wall 126 at open end 124 of compartment 120. The compressively abutting contact may be accomplished by specifying L₁<L₂, as discussed previously in relation to the embodiment in FIGS. 7 and 8. Also, as in the previously discussed embodiment, the positions of the complementary groove and ridge segment snap-fit elements can be interchanged. Moreover, a continuous ridge, such as ridge 128′ (as shown in FIG. 12) can be substituted for ridge segments 128 for use with the continuous groove 122. Or, groove segments (not shown) could be substituted for use with ridge segments 128, as previously discussed.

First capsule compartment 102, including linker element 110, and second capsule compartment 120, together with their respective snap-fit elements, can be formed by injection molding. Also, depressions in the walls of compartments 102 and 120 can be provided to accommodate injection material overflow and retain a smooth overall dosage form surface, such as depressions 134 at the closed compartment ends (see FIG. 11).

In accordance with yet another aspect of the present invention, as disclosed and claimed herein, a linker is provided for connecting two dosage form units from the group including capsule compartments and closure caps, the units having respective open ends. More specifically, the linker includes a substantially cylindrical tube having an interior and a longitudinal axis, and also an outer surface and opposed axial ends. A raised band is positioned on the outer tube surface between the opposing actual tube ends and extends circumferentially around the tube outer surface. The raised band has opposed side surfaces configured for a compressive abutting contact with the open ends of the dosage form units. Also, snap-fit elements are located on the tube outer surface between the raised end and each of the tube axial ends. Preferably, the snap-fit elements are selected from circumferential grooves, groove segments, ridges, and ridge segments. The linker further includes a wall configured and dimensioned to close the tube interior, with the wall being positioned along the axis at a location between and including the axial tube ends.

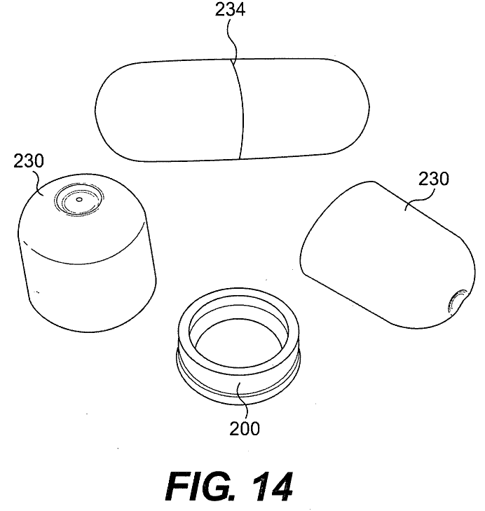

As embodied herein and with initial reference to FIG. 13, linker 200 includes a generally cylindrical tube 201 having outer wall 202 and axis 204, and defining interior 206. Linker 200 has opposed axial tube ends 208, 210, and a raised circumferential band 212 on outer surface 214 of wall 202 between tube ends 208, 210. Groove-type snap-fit elements 216, 218 are formed on outer surface 214 between band 212 and tube ends 208, 210, respectively, for engaging complementary snap-fit elements on capsule compartments or cap type units to be connected (not shown in FIG. 13) in the manner discussed in relation to the embodiment of FIGS. 1 and 4-8. Linker 200 also includes wall member 220 closing off interior 206 from flow through tube 201. Wall 220 can be located at any location between and including the two ends 208, 210 including approximately mid-way therebetween, as shown in FIG. 13. A location of wall 220 at the tube end 210 is shown dashed in FIG. 13. The position of wall 220 can be as required, e.g., for forming convenience, such as by injection molding, or for using the interior 206 to augment drug holding capacity of one or the other (or both) attached capsule compartments. It is contemplated that linker 200 may be a less preferred linker for holding a drug substance in one attached capsule compartment separate from a drug substance in another attached capsule compartment of the dosage form as illustrated in FIG. 14, as compared to linker 10 in the embodiment of FIG. 1.

While raised band 212 is shown in FIG. 13 as having parallel opposed sides 212a and 212b for abutting contact with capsule compartment ends, FIGS. 14 and 15 depict a variation where raised band 212′ is wedge-shaped, having sides 212a′ and 212b′ forming oblique angles with outer surface 214. This linker variation 200′ allows the use of capsule compartments 230 having walls 230 with tapered ends 232 for abutting not only raised band 212′, but each other. This construction would provide a single juncture or seam, namely a circumferential seam 234 depicted in FIGS. 14 and 15. A single seam feature may increase the integrity of the dosage formed by reducing the potential for seam separation, with respect to the two-seam construction of the band 212 in FIG. 13, and thus may be preferred in some applications. Also, cap type units, such as cap 250 shown dashed in FIG. 15, can be used instead of one or both of the capsule compartments with the linker depicted in FIGS. 13-15.

However, in other applications, such as where two capsule compartments having different outside diameters are to be connected, such as, example, a 0.4 mm capsule and a 0.5 mm capsule, a linker providing two seams may be advantageous. For example, as depicted in FIG. 16, linker 200″ includes raised band 212″ having an axially tapered top surface 236 to provide a smooth transition between the outer surfaces of capsule compartment 238, having a diameter D₁ adjacent open end 240, and capsule compartment 242, having a smaller diameter D₂ adjacent open end 244. Abutting contact between raised band sides 246, 248 and capsule ends 240 and 244, respectively, may provide two circumferential seams but with a band top surface 236 providing an inclined, transitional surface. Also, while linker 200″ is shown with end wall 220 adjacent the smaller diameter capsule 242, linker 200″ could be configured with wall 200 adjacent the large diameter capsule 238, or at any other convenient location in-between.

In accordance with still another aspect of the present invention, as embodied and described herein, a linker is disclosed for connecting and separately sealing two capsule compartments. The linker includes a first generally cylindrical linker part having an axis and opposed longitudinal ends. The first linker part, which can be labeled a “female” part, has an outer surface with a diameter, an inner surface with a diameter, and a closed wall. The outer surface includes a first snap-fit element configured for engaging a complementary snap-fit element on a capsule compartment, and the inner surface includes another snap-fit element. The linker further includes a second generally cylindrical linker part, which can be labeled a “male” linker part, that has an axis, opposed longitudinal ends, and includes a closed wall. The second linker part has a large diameter portion adjacent one end of the second part and a small diameter portion adjacent, and extending axially from, the other end of the second part. The large and small diameter portions have respective outer surfaces, with the outer surface of the large diameter portion including a snap-fit element configured for engaging a complementary snap-fit element on a capsule compartment and the outer surface of the small diameter portion including a snap-fit element complementary to the another snap-fit element on the inner surface of the first linker part. The another snap-fit element and the small diameter portion snap-fit element are engageable to releaseably connect the first and second linker parts. Also, the first and second closed walls are configured to substantially seal off the first and second capsule compartments when the linker parts are engaged with the respective capsule compartments.

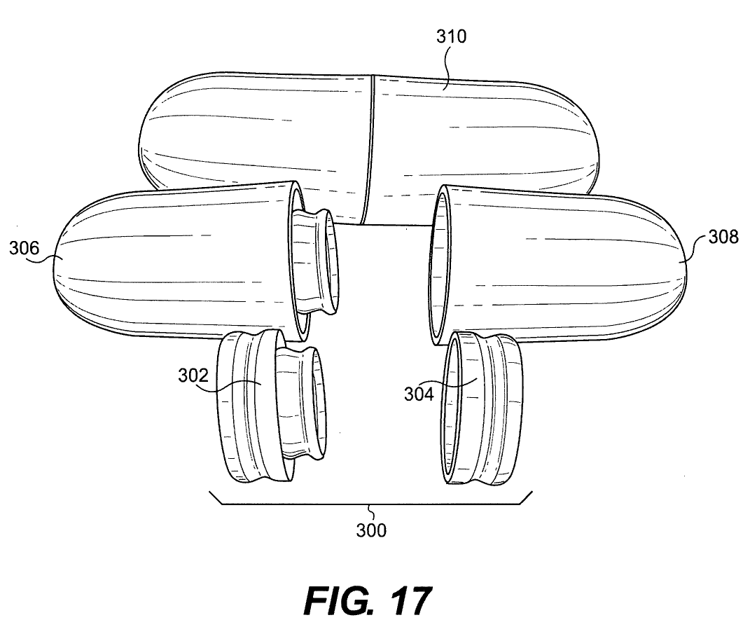

As embodied herein with initial reference to FIG. 17, linker 300 includes a “male” linker part 302 and a “female” linker part 304. Each of parts 302, 304 is configured to be separately engageable with dosage form units, such as capsule compartment 306 (shown connected to male part 302 in FIG. 17) and capsule compartment 308 (shown connected to female part 304). As will be discussed below, the connections between linker parts 302, 304 and respective capsule compartments 306 and 308 are snap-fit connections preferably using complementary snap-fit elements selected from circumferential grooves, groove segments, ridges, and ridge segments. Also, the interconnection between linker parts 302 and 304, to be discussed in more detail hereinafter to provide a completed dosage form, such as dosage form 310 depicted in FIG. 17, is a snap-fit connection, using appropriate, complementary snap-fit elements.

It also may be preferred that the connection between the separate linker parts 302, 304, and respective capsule compartments 306, 308 be sealing connections such that “sets” of prefilled dosage form modules can be prepared, transported separately, and selectively assembled with other modules holding drug substances with or without different compositions and/or release properties. The sealed modules comprising a capsule compartment with an engaged linker part 302 or 304 could also be assembled with modules using capsule compartments with different release properties. As would be understood by those skilled in the art, the interconnection of linker parts 302 and 304 during assembly need not be a sealing connection, although it is highly preferred to be a connection providing abutting contact to foster dosage form integrity and a continuous overall surface, as discussed previously in relation to the other embodiments.

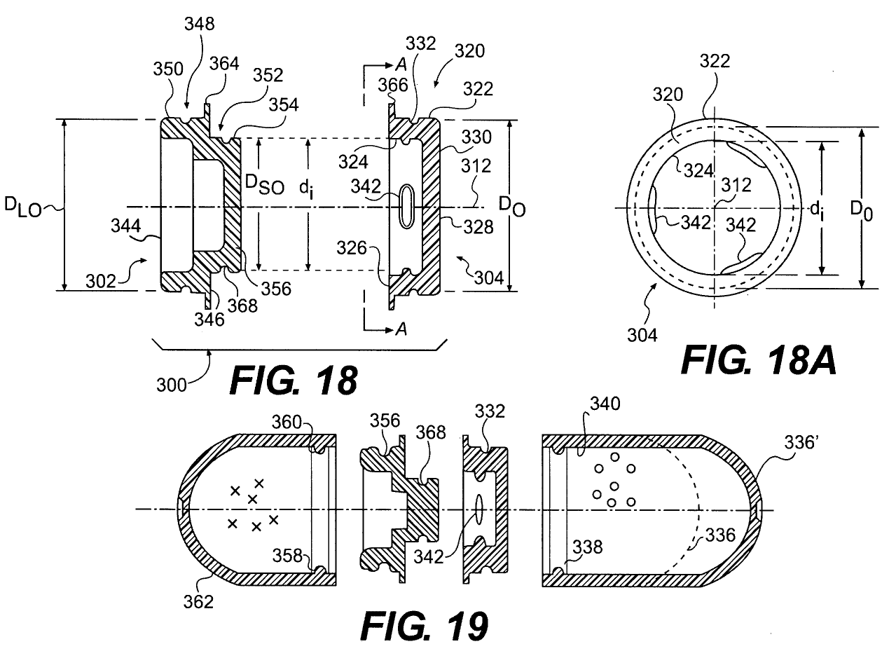

FIGS. 18 and 18a show details of the linker parts 302 and 304 of the two-part linker 300. As shown, both linker parts 302, 304 are generally cylindrical and substantially symmetric about axis 312. Female linker part 304 has outer wall 320 with outer surface 322 having a diameter Dₒ, inner surface 324 with diameter dᵢ, and opposed longitudinal ends 326, 328. Female linker part 304 also includes wall 330 closing off the interior and preventing flow through female linker part 304. A snap-fit element in the form of continuous circumferential groove 332 is formed on outer surface 322 for engagement with a dosage form unit to be connected, such as capsule compartment 336 (FIG. 19) that has continuous ridge 338 on inner capsule surface 340. Female linker part 304 also has a snap-fit element on inner surface 324, namely three ridge segments 342 (FIG. 18A) angularly spaced about axis 312 for engagement with a complementary snap-fit element on male linker part 302, details of which follow.

Male linker part 302 includes opposed longitudinal ends 344, 346, with end 344 including a “large” diameter portion 348 having an outer surface 350 of diameter D_{Lo}. Male linker part 302 also has a “small” diameter portion 352 adjacent to and extending axially from male linker end 346. The “small” linker part 352 includes outer surface 354 with a diameter Dₛₒ. Closing wall 356 is provided to prevent flow through male linker part 302, and is positioned at the end of portion 352 for injection molding convenience. However, closing wall 356 could be positioned at male linker end 344 or anywhere in-between.

As with female linker part 304, male linker part 302 has a snap-fit element for engaging a complementary snap-fit element on a capsule compartment or other dosage form unit to be connected. In particular, as shown in FIGS. 18 and 19, groove 356 is provided on outer surface 350 of male linker 302 and is intended to engage circumferential ridge 358 disposed on inner surface 360 of capsule compartment 362 (see FIG. 19).

Preferably, each of female linker part 304 and male linker part 302 include a raised band (which may be construed as axially segmented parts of a single band), such as raised band 364 on outer surface 350 of male linker part 302 and raised band 366 on the outer surface 322 of female linker part 304. Raised bands 364 and 366 are intended to provide structure against which the open ends of capsule compartments (or other dosage form units) can abuttingly contact. As stated previously in relation to the other embodiments, use of circumferential ridges and grooves as snap-fit elements together with a raised band can provide two circumferential lines of sealing contact, one between the contacting snap-fit elements and another between capsule ends and the raised band, to reduce leakage. However, two-part linker constructions having linker parts similar to parts 302, 304 but which include only a single raised band, on either the male or female linker part, or with no raised band at all are contemplated. In each such case, the capsules and snap-fit elements could be configured and dimensioned such that the engaging force provided by the snap-fit elements connecting the male and female linker parts could provide abutting contact with the single raised band, in the first case, or abutting contact between opposed open ends of the capsule compartments in the latter case. However, such constructions are not presently preferred.

Male linker part 302 further includes groove-type snap-fit element 368 for engagement with ridge segments 342 on female part 304. While three ridge segments 342 are shown (FIG. 18A), fewer or more can be used, as well as a continuous circumferential ridge. The configurations of groove 368 and ridge segments 342 as well as spacings of groove 368 and ridge segments 342 from end 346 and end 326 respectively, should preferably be arranged to provide compressive abutting contact between the ends, including the abutting sides of raised bands 364 and 366. This compressive abutting contact may increase dosage form integrity and respective sealing of the capsule units. Also, if a raised band configuration such as depicted in FIG. 15 is used (but in a “split” construction between female linker part 304 and male linker part 302) a single “external” seam would result, reducing discontinuities in the overall exterior surface for the dosage form. However, a raised band construction providing a “transition” dosage form surface such as depicted in FIG. 16 may be desired for dosage forms requiring capsules of different diameters, that is, dosage forms asymmetric in the diameter dimension. In such a case, the diameter D_{Lo} of outer surface 350 of male part 302 and/or diameter Dₒ of outer surface 322 of female part 304, together with the associated snap-fit grooves may have to be sized to reflect the different diameters of the inner surfaces of the specific capsule compartments to be used. Also, the dimensions of raised bands 364 and 366 may be changed and configured with top surfaces such that, when bands 364 and 366 are abutted, they provide an axial taper similar to the single band version of a tapered raised band shown in FIG. 16. Of course, length-wise asymmetric dosage forms can be readily achieved with the construction shown in FIGS. 17 and 19. For example, a shortened version of capsule 336 shown dashed as 336′ in FIG. 19. Alternatively, a shorter (or longer) version of capsule 362 is also contemplated.

Each of the capsule compartments, closure caps, linkers, and linker parts may be made of a transitional polymer and may comprise the same or different polymer. A transitional polymer is a polymer that changes shape, form, or structure within a gastro-intestinal environment, e.g., dispersible, dissolvable, disintegrable, breachable, swellable, partially or completely soluble, fracturable, or otherwise changeable when exposed to stomach pH and/or in intestine pH. Suitable polymers include: polyvinyl alcohol (PVA), natural polymers (such as polysaccharides like pullulan, carrageenan, xanthan, chitosan or agar gums), polyethylene glycols (PEG), polyethylene oxides (PEO), mixtures of PEGS and PEOS, hydroxypropylmethylcellulose (HPMC), methylcellulose, hydroxyethylcellulose, hydroxyethyl methylcellulose, hydroxypropylcellulose, methacrylic acid copolymer (such as Eudragit E™, Eudragit L™ and/or Eudragit S™), ammonium methacrylate copolymers (such as Eudragit RL™ and/or Eudragit RS™), carboxymethylcellulose, povidone (polyvinyl pyrrolidone), polyglycolysed glycerides (such as Gelucire 44/14™, Gelucire 50/02™, Gelucire 50/13™ and Gelucire 53/10™), carboxyvinyl polymers (such as Carbopols™), polyoxyethylene-polyoxypropylene copolymers (such as Poloxamer 188™), and acrylic and/or methacrylic acid-based polymers. The Eudragit polymers discussed above for example are extrudable and may for example be plasticised with e.g. triethyl citrate, or glyceryl monostearate.

Preferred polymers are orally ingestible polymers and include hydroxypropyl methylcellulose acetate succinate (HPMC-AS), polyvinyl alcohol, hydroxypropyl methyl cellulose, and other cellulose-based polymers. Preferred polymers also include polymer materials which preferentially dissolve or disintegrate at different points in the digestive tract. Such polymers include the known acrylic and/or methacrylic acid-based polymers which are transitional in intestinal fluids, e.g. the Eudragit series of commercially available polymers. Examples of these include Eudragit E™, such as Eudragit E 100™ or Eudragit 4135F™, which preferentially dissolves in the more acid pH of the stomach, or enteric polymers such as Eudragit L™ and/or Eudragit S™ which preferentially dissolve in the more alkaline pH of the intestine, and preferred polymers also include polymers which dissolve slowly, e.g. at a predetermined rate in the digestive tract, such as Eudragit RL™ e.g. Eudragit RL 100™, and/or Eudragit RS e.g. Eudragit R100™, and/or blends of such Eudragit™ polymers.

The polymers may include other substances to modify their properties and to adapt them to various applications, including, for example, the following general classes of substances: surfactants, such as Polysorbate 80™, sodium lauryl sulphate, and Polyoxyl 40™ hydrogenated castor oil; absorption enhancers, such as Labrasol™, Transcutol™; glidants, such as stearyl alcohol, talc, magnesium stearate, silicon dioxide, amorphous silicic acid, fumed silica, Simeticone™; plasticizers, such as triethyl citrate, acetyl triethyl citrate, tributyl citrate, acetyl tributyl citrate, glyceryl monostearate, diethyl phthalate, dibutyl phthalate, propylene glycol, triacetin and castor oil; substances for release modification, such as ethyl cellulose and cellulose acetate phthalate; disintegrants, such as sodium starch glycollate, croscarmellose sodium, crospovidone (cross-linked polyvinyl pyrrolodone), coloring agents, flavoring agents and sweetening agents.

It is contemplated that in addition and/or as an alternative to the full or partial circumferential beads and grooves, a linker or a linker part may be connected to a capsule compartment, a closure cap, and/or another linker part via a threaded screw-type connection. It is also contemplated that if such a threaded screw-type connection is utilized, a first one of the capsule compartment, closure cap, linker, or linker part may include an external threaded element and a second one of the capsule compartment, closure cap, linker, or linker part may include an internal threaded element configured to be complementary to and engagable with the external threaded element. It is further contemplated that if such a threaded screw-type connection is utilized, the material from which the capsule compartment, closure cap, linker, or linker part may be strengthened via material re-work, additives to the polymer, and/or increased tolerances with respect to the mold or injection process as is known in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A linker for connecting with one or more dosage form units from a group including capsule compartments and closure caps, the linker being configured to include a drug substance and comprising:
a solid drug substance in tablet form, the tablet having a longitudinal axis and being substantially cylindrical with opposed axial end faces;
a jacket radially confining the tablet, the jacket having an outer wall with longitudinal ends, at least one of the jacket longitudinal ends being open for dispensing the drug substance from the respective end face,
wherein the outer wall further includes a snap-fit element located adjacent at least one jacket longitudinal end.

2. The linker as in claim 1, further including the jacket having a closed end wall adjacent to one or the other of said jacket longitudinal ends for substantially blocking tablet drug substance from dispensing from the respective tablet axial end face.

3. The linker as in claim 1, wherein the snap-fit element is selected from circumferential grooves, groove segments, ridges, and ridge segments.

4. The linker as in claim 1, further including a raised circumferential band on the jacket outer wall between the jacket longitudinal ends, the band including a side surface configured for axial abutting contact with the dosage form unit.

5. The linker as in claim 1, wherein at least one of the tablet end faces is exposed and configured to extend axially beyond a respective jacket longitudinal end.

6. The linker as in claim 1, wherein the tablet has a recessed land around the perimeter of at least one tablet axial end faces, and wherein the jacket includes inwardly directed outer wall end edges that engage the recessed land.

7. The linker as in claim 1, wherein the tablet has two or more cylindrical parts in axially abutting contact, wherein the parts are comprised of different drug substances having different compositions and/or different release characteristics.

8. The linker as in claim 4, wherein the linker connects two dosage form units, wherein the raised band has two opposed side surfaces for abuttingly contacting the two units, and wherein the raised band has a top surface configured and dimensioned to transition between outer surfaces of the two dosage form units.

9. The linker as in claim 1, wherein the jacket comprises a material selected from the group consisting of hydroxypropyl cellulose, hydroxypropyl methylcellulose acetate succinate, polyvinyl alcohol, hydroxypropyl methyl cellulose, and acrylic or methacrylic acid-based polymers.

10. A multicomponent dosage form comprising:
a linker unit including
a solid drug substance in tablet form, the tablet having a longitudinal axis and being substantially cylindrical with opposed longitudinal end faces;
a jacket radially confining the tablet, the jacket having an outer wall with longitudinal ends and a raised circumferential band between the jacket longitudinal ends, and
wherein the outer wall further includes a snap-fit element located adjacent at least one longitudinal end, the element being selected from circumferential grooves, groove segments, ridges and ridge segments; and
at least one dosage form unit selected from the group including capsule compartments and closure caps, the dosage form unit having an open end connected to the one jacket longitudinal end, the unit having a snap-fit element complementary to and configured to engage the snap-fit element adjacent the one jacket longitudinal end.

11. The multicomponent dosage form as in claim 10, wherein the linker unit snap-fit element is a circumferential groove and the dosage form unit complementary snap-fit element is a circumferential ridge positioned on an inner wall of the dosage form unit.

12. The multicomponent dosage form as in claim 10, wherein the jacket also has a closed end wall.

13. The multicomponent dosage form of claim 12, wherein the linker jacket and closed wall each comprise a material selected from the group consisting of hydroxypropyl cellulose, hydroxypropyl methylcellulose acetate succinate, polyvinyl alcohol, hydroxypropyl methyl cellulose, and acrylic or methacrylic acid-based polymers.

14. The multicomponent dosage form of claim 10,
wherein the dosage form unit has an inner surface proximate the open end, the dosage form unit complementary snap-fit element being located on a dosage unit inner surface, and
wherein the snap-fit element of the jacket and the complementary snap-fit element of the dosage form unit are configured and longitudinally spaced to provide compressive abutting contact between the jacket raised circumferential band and the open end of the dosage form unit, when the respective snap-fit and complementary snap-fit elements are engaged.

15. The multicomponent dosage form as in claim 10, wherein the raised circumferential band has a side configured for contacting the open end of the dosage form unit, and a longitudinal spacing between the raised band side and the snap-fit element on the longitudinal jacket end is less than a longitudinal spacing between the dosage form unit open end and the complementary snap-fit element on the inner wall of the connected dosage form unit.

16. The multicomponent dosage form of claim 10, wherein the linker unit snap-fit element is a circumferential groove and the dosage form unit complementary snap-fit element is a circumferential ridge.

17. The multicomponent dosage form of claim 16, wherein the circumferential groove has a generally curved or wedge-shaped axial cross-section.

18. A multicomponent dosage product comprising:
a linker unit including
a solid drug substance in tablet form, the tablet having a longitudinal axis and being substantially cylindrical with opposed longitudinal end faces;
a jacket radially confining the tablet, the jacket having an outer wall with opposed longitudinal ends and a raised circumferential band between the jacket ends,
wherein the outer wall further includes a snap-fit element located between the raised circumferential band and one of the longitudinal ends, the snap-fit element being selected from circumferential grooves, groove segments, ridges, and ridge segments; and
a first capsule compartment having an open end connected to the linker unit at the one jacket longitudinal end, the first capsule compartment having a snap-fit element complementary to and engaging the snap-fit element of the one jacket longitudinal end,
wherein the first capsule compartment contains a first drug substance different in composition and/or in release characteristics from the tablet drug substance.

19. The multicomponent dosage form as in claim 18, wherein a closure cap is connected to the linker unit at the other jacket longitudinal end, the closure cap having an open end and a snap-fit element complementary to and engaging another linker unit snap-fit element adjacent the other jacket longitudinal end.

20. The multicomponent dosage form as in claim 18, further including the jacket having a closed end wall adjacent at least one jacket longitudinal end.

21. The multicomponent dosage form of claim 18, wherein the jacket is formed from a material selected from the group consisting of hydroxypropyl cellulose, hydroxypropyl methylcellulose acetate succinate, polyvinyl alcohol, hydroxypropyl methyl cellulose, and acrylic or methacrylic acid-based polymers.

22. The multicomponent dosage form as in claim 19, wherein the linker unit snap-fit elements are circumferential grooves, and the complementary snap-fit elements of the first capsule compartment and closure cap are circumferential ridges positioned on respective inner walls of the first capsule compartment and the closure cap.

23. The multicomponent dosage form of claim 19, wherein the linker unit snap-fit elements and complementary snap-fit elements of the closure cap and first capsule compartment are configured and spaced to provide compressive abutting contact between the raised circumferential band and the open ends of the first capsule compartment and the closure cap.

24. The multicompartment dosage form as in claim 19, wherein the axial lengths between the snap-fit elements and the raised band on the linker unit are respectively less than the axial lengths between the snap-fit elements and open ends of the first capsule compartment and closure cap.

25. The multicompartment dosage form as in claim 19, wherein the raised outer band has a top surface configured and dimensioned to provide a transition between outer surfaces of the first capsule compartment and closure cap.

26. The multicomponent dosage form as in claim 18, wherein the raised band includes one or more apertures configured to allow dispersal of the tablet drug substance radially past through the jacket.

27. The multicomponent dosage form of claim 18, further including a second capsule compartment having an open end connected to the linker unit at the other jacket longitudinal end, the second capsule compartment having a snap-fit element complementary to and engaging a snap-fit element adjacent the other jacket longitudinal end, the second capsule compartment containing a second drug substance different in composition and/or release characteristics from the first drug substance or from the tablet drug substance.

28. The multicomponent dosage form of claim 27, wherein a longitudinal length of the second capsule compartment is greater than a longitudinal length of the first capsule compartment.

29. The multicomponent dosage form of claim 27, wherein a diameter of the second capsule compartment is greater than a diameter of the first capsule compartment.

30. The multicomponent dosage form of claim 27, wherein the linker unit snap-fit elements and complementary snap-fit elements of the first and second capsule compartments are configured and spaced to provide compressive abutting contact between the raised circumferential band and the open ends of the first and second capsule compartments.

31. The multicompartment dosage form as in claim 27, wherein the axial lengths between the snap-fit elements and the raised band on the linker unit are respectively less than the axial lengths between the complementary snap-fit elements and open ends of the first and second capsule compartments.

32. The multicomponent dosage form in claim 27, wherein the snap-fit elements of the first capsule compartment and second capsule compartment are circumferential ridges having a generally rounded axis cross-sectional shape, and wherein the linker snap-fit elements are circumferential grooves having a generally rounded or wedge-shaped cross-section.

33. The multicomponent dosage form as in claim 27, wherein the outer surfaces of the first and second capsule compartments have different diameters and wherein the top surface of the raised circumferential band tapers toward the axis in the direction of the capsule compartment with the smaller diameter.

34. The multicomponent dosage form as in claim 27, wherein the raised circumferential band is configured and dimensioned to provide abutting contact between the ends of the first and second capsule compartments.

35. The multicomponent dosage form as in claim 34, wherein the raised band is wedge shaped.

36. A linker for connecting two dosage form units from the group including capsule compartments and closure caps, the dosage form units each having an open end, the linker comprising:
a substantially cylindrical tube having an interior and a longitudinal axis, the tube also having an outer surface and opposed axial ends;
a raised band disposed on the tube outer surface, the band being located between the opposed axial tube ends and extending circumferentially around the tube outer surface, the raised band having opposed side surfaces configured for compressive abutting contact with the open ends of the dosage form units;
snap-fit elements located on the tube outer surface between the raised band and a respective tube axial end, the snap-fit elements being selected from circumferential grooves, groove segments, ridges, and ridge segments; and
a wall configured to close the tube interior, the wall being positioned along the axis at a location at one of, or between, the tube axial ends.

37. A dosage form comprising:
a linker comprising a substantially cylindrical tube having an interior and a longitudinal axis, the tube also having an outer surface and opposed axial ends;
a raised band positioned on the outer surface, the band being located between the opposed axial tube ends and extending circumferentially around the tube outer surface, the raised band having opposed side surfaces oriented substantially perpendicular to the axis;
snap-fit elements located on the tube outer surface between the raised band and a respective tube axial end, the snap-fit elements being selected from circumferential grooves, groove segments, ridges, and ridge segments;
a wall configured and dimensioned to close the tube, the wall being positioned along the axis one of, or between, the tube axial ends; and
a pair of dosage form units selected from capsule compartments and closure caps connected to the linker axial ends, each of said units having a complementary snap-fit element formed on a respective inner unit surface adjacent an open end and being engaged with a respective snap-fit element on the linker,
wherein the configuration of the snap-fit elements and spacing from the raised band on the linker and the configuration of the complementary snap-fit elements and spacing from the open ends on the dosage form units provides a compressive abutting contact between the open ends of the dosage form units and the respective sides of the raised band.

38. A linker for connecting and separately sealing first and second capsule compartments, the linker comprising:
a first generally cylindrical linker part having an axis and opposed longitudinal ends, the first part also having an outer surface with a diameter, an inner surface with a diameter, and a closed wall, the outer surface including a first snap-fit element configured for engaging a complementary snap-fit element on the first capsule compartment, and the inner surface including another snap-fit element, the first snap-fit element and the another snap-fit element being selected from circumferential grooves, groove segments, ridges, and ridge segments; and
a second generally cylindrical linker part having an axis and opposed longitudinal ends, the second linker part having a large diameter portion adjacent a first end of the second part and a small diameter portion adjacent and extending axially from a second end of the second part, the large and small diameter portions having respective outer surfaces, the second part also having a closed wall,
wherein the outer surface of the large diameter portion includes a second snap-fit element configured for engaging a complementary snap-fit element on the second capsule compartment, the second snap-fit element being selected from circumferential grooves, groove segments, ridges, and ridge segments,
wherein the outer surface of the small diameter portion contains a snap-fit element selected from circumferential grooves, groove segments, ridges, and ridge segments complementary to the another snap-fit element on the inner surface of the first linker part.

39. A linker for connecting and substantially separately sealing a first and a second capsule compartment comprising:
a first linker part including a first axial end and a second axial end and including first and second snap-fit elements disposed adjacent a respective one of the first and second axial ends thereof;
a second linker part include a first axial end and a second axial end and including third and fourth snap-fit elements disposed adjacent a respective one of the first and second axial ends thereof;
wherein the first snap-fit element is configured to selectively engage a complementary snap-fit element on the first capsule compartment, the fourth snap-fit element is configured to selectively engage a complementary snap-fit element on the second capsule compartment, and the second snap-fit element is configured to be complementary to and selectively engageable with the third snap-fit element.

40. An apparatus for containing a drug substance comprising:
a linker element having a generally cylindrical outer wall, a first snap-fit element disposed on an outer surface thereof and at least one closed axial end wall; and
a capsule compartment having a generally cylindrical outer wall, a first complementary snap-fit element disposed on an inner surface thereof, a closed axial end and an open axial end;
wherein the first snap fit element and first complementary snap-fit element are selectively engageable; and
wherein the linker element is configured to substantially seal the drug substance within a region defined by at least the linker outer wall and the closed axial end of the capsule compartment and the closed axial end wall of the linker element when the first snap-fit element and the first complementary snap-fit element are engaged.

41. A dosage form comprising:
a first generally cylindrical capsule compartment having an axis, a closed end, and an open end, the open end including an integral, annular linker member extending axially outward of the open end and having an outer surface with a first snap-fit element located a distance L₁ from the capsule compartment open end;
a second generally cylindrical capsule compartment having an axis, a closed end, and an open end with an inner surface, the second capsule compartment having a second snap-fit element complementary to the first snap-fit element, the second snap-fit element being located on the inner surface and spaced a distance of L₂ from the second capsule open end,
wherein L₂>L₁ and the first and second snap-fit elements are engageable to connect the first and second capsule compartments with the respective open ends being in compressive abutting contact with each other.
